(19) United States
(12) Patent Application Publication
(10) Pub. No.: US 2026/0112467 A1
(43) Pub. Date: Apr. 23, 2026

(54) SYSTEM AND METHOD FOR AUTOMATED HEALTH RECORD GENERATION

(71) Applicant: Acuity Holding Corp., Burnaby (CA)

(72) Inventors: Michael Charles WOOD, Vancouver (CA); Miguel PALOMINO MADRID, Vancouver (CA)

(73) Assignee: Acuity Holding Corp., Burnaby (CA)

(21) Appl. No.: 19/334,767

(22) Filed: Sep. 19, 2025

Related U.S. Application Data
(60) Provisional application No. 63/696,639, filed on Sep. 19, 2024.

(51) Int. Cl. G16H 10/60; G10L 15/16; G10L 15/26

(52) U.S. Cl. CPC G16H 10/60; G10L 15/16; G10L 15/26

(57) ABSTRACT

A system and computer-implemented method of automatically generating a record, in a user-oriented record-keeping application, based on vocalized expression. The method includes receiving an audio signal indicative of the vocalized expression; generating a first transcript using a first transcription model based on the audio signal; generating a second transcript using a second transcription model based on the audio signal, independently of the first transcription model; determining a third transcript based on the first and second transcripts by using a large language model; using the third transcript and a machine learning model to determine data suitable for generating the record; and operating the record-keeping application using a desktop automation tool based on the data to generate the record.

### CROSS-REFERENCE TO RELATED APPLICATIONS

Any and all priority claims identified in the Application Data Sheet, or any correction thereto, are hereby incorporated by reference under 37 CFR 1.57.

This application claims the benefit of U.S. Provisional Application No. 63/696,639, filed on 19 Sep. 2024.

Each of the aforementioned applications is incorporated by reference herein in its entirety, and each is hereby expressly made a part of this specification.

### TECHNICAL FIELD

The disclosure relates generally to patient records, and more particularly to a system and method for automated generation of patient records.

### BACKGROUND

Health professionals spend a considerable amount of time generating and maintaining patient records. For each interaction with a patient, a summary of the interaction is drafted by the health professional, often with the aid of notes taken during the interaction. In addition, specific examination details are also recorded. For example, a dentist may create an odontogram that records the condition of each of a patient's teeth. For example, an odontogram may include details of tooth sensitivity, tooth cavities, tooth filling, and so on. Such health records help with diagnosis and treatment planning. This is particularly true for longitudinal care.

It is now standard practice to use computerized systems to generate health records. Such computerization is desired for a variety of reasons, including to standardize records so that health records may be easily compared to each other, e.g. for conducting large-scale analysis of patient data for public health reasons.

The most popular computerized systems generally include software that provides dentists with either click-through menus with fillable forms to capture specific details of a patient interaction or static fillable forms that can be submitted for record generation. Such systems have improved accessibility and searchability of health records. They have also reduced the amount of physical records and manual work needed to be done by health professionals and their assistants. Nevertheless, organizing and entering data into these systems still poses a considerable time burden upon health professionals. Improvement is desired.

### SUMMARY

It is found that a record of a patient interaction may be generated in an efficient manner by processing audio of the patient interaction to generate data suitable for entering into a preexisting or legacy software package. The data is then entered into the legacy software package via a desktop automation tool. Advantageously, healthcare professionals continue to be able to use preexisting systems that they are familiar with, with the proposed technology being formed as an additional layer thereon.

Generating data suitable for entry into preexisting or legacy software based on recorded audio can be challenging. For example, it is required to identify speakers in the recording, generate a coherent summary narrative from a back-and-forth interaction between the patient and the doctor, identify specific parameters mentioned by the healthcare professional and/or patient, and so on. It is found that such data may be effectively generated by using a plurality of transcription models, each of which can independently generate verbal transcripts of vocalized expressions in audio, to generate a plurality of transcripts. This plurality of transcripts is then processed via a plurality of independent large language models (LLMs), such as ChatGPT™, Claude™, or Llama 3™ to generate data to be used by the desktop automation tool to enter into the preexisting or legacy software.

Aspects disclosed herein are found to be particularly advantageous for dental professionals.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

There is disclosed a computer-implemented method of automatically generating a patient record. The computer-implemented method also includes receiving an audio signal indicative of the vocalized expression; generating a first transcript using a first transcription model based on the audio signal; generating a second transcript using a second transcription model based on the audio signal, independently of the first transcription model; determining a third transcript based on the first and second transcripts by using a large language model; using the third transcript and a machine learning model to determine data suitable for generating the patient record; and operating the record-keeping application using a desktop automation tool based on the data to generate the patient record.

Embodiments include corresponding computer systems, apparatus, and one or more computer storage devices, such as non-transitory computer-readable media, having recorded thereon computer programs configured to cause performance or execution of the steps or actions of the methods.

Implementations may include one or more of the following features. The method where determining the third transcript based on the first and second transcripts by using a large language model includes determining the third transcript based on the first and second transcripts by using a plurality of independent large language models. Determining the third transcript based on the first and second transcripts by using a plurality of independent large language models includes generating a plurality of candidate transcripts using the plurality of independent large language models, each of the plurality of candidate transcripts being uniquely associated with a corresponding one of the plurality of independent large language models; and generating the third transcript based on the plurality of candidate transcripts. Generating the third transcript based on the plurality of candidate transcripts includes receiving a user input responsive to the plurality of candidate transcripts, and modifying the plurality of candidate transcripts based on the user input to generate the third transcript.

Generating the third transcript based on the plurality of candidate transcripts includes using a supervised learning model to process the plurality of candidate transcripts to generate the third transcript, the method may include updating the supervised learning model based on the user input. Generating the third transcript based on the plurality of candidate transcripts includes using a supervised learning model to process the plurality of candidate transcripts to generate the third transcript. The large language model is a first large language model, and the machine learning model is a second large language model independent of the first large language model. Generating the first transcript using the first transcription model based on the audio signal includes generating a first candidate transcript using the first transcription model based on the audio signal, receiving a first user input responsive to the first candidate transcript, and modifying the first candidate transcript based on the first user input to generate the first transcript; and generating the second transcript using the second transcription model based on the audio signal, independently of the first transcription model, includes generating a second candidate transcript using the second transcription model based on the audio signal, receiving a second user input responsive to the second candidate transcript, modifying the second candidate transcript based on the second user input to generate the second transcript. The first transcription model includes a first machine learning model, the method may include: updating the first machine learning model based on the first user input. Determining the third transcript based on the first and second transcripts by using the large language model includes generating a first candidate transcript using the first transcription model based on the audio signal, receiving a first user input responsive to the first candidate transcript, and modifying the first candidate transcript based on the first user input to generate the first transcript. Operating the record-keeping application using a desktop automation tool based on the data to generate the patient record includes storing the patient record in a database. Implementations of the described techniques may include hardware, a method or process, or computer software on a computer-accessible medium.

There is disclosed a system for automatically generating a patient record. The system also includes a processor; and computer-readable memory coupled to the processor and storing processor-executable instructions that, when executed, configure the processor to: generate a first transcript using a first transcription model based on an audio signal indicative of the vocalized expression, generate second transcript using a second transcription model based on the audio signal, independently of the first transcription model, determine a third transcript based on the first and second transcripts by using a large language model, use the third transcript and a machine learning model to determine data suitable for generating the patient record, and operate the record-keeping application using a desktop automation tool based on the data to generate the patient record.

Implementations may include one or more of the following features. The system where to determine the third transcript based on the first and second transcripts by using a large language model includes to determine the third transcript based on the first and second transcripts by using a plurality of independent large language models. To determine the third transcript based on the first and second transcripts by using a plurality of independent large language models includes to generate a plurality of candidate transcripts using the plurality of independent large language models, each of the plurality of candidate transcripts being uniquely associated with a corresponding one of the plurality of independent large language models; and generate the third transcript based on the plurality of candidate transcripts. To generate the third transcript based on the plurality of candidate transcripts includes to receive a user input responsive to the plurality of candidate transcripts, and modify the plurality of candidate transcripts based on the user input to generate the third transcript. To generate the third transcript based on the plurality of candidate transcripts includes to use a supervised learning model to process the plurality of candidate transcripts to generate the third transcript, and the processor-executable instructions, when executed, further configure the processor to: update the supervised learning model based on the user input. To generate the third transcript based on the plurality of candidate transcripts includes to use a supervised learning model to process the plurality of candidate transcripts to generate the third transcript. The large language model is a first large language model, and the machine learning model is a second large language model independent of the first large language model. To generate the first transcript using the first transcription model based on the audio signal includes to generate a first candidate transcript using the first transcription model based on the audio signal, receive a first user input responsive to the first candidate transcript, and modify the first candidate transcript based on the first user input to generate the first transcript; and generate the second transcript using the second transcription model based on the audio signal, independently of the first transcription model, includes generate a second candidate transcript using the second transcription model based on the audio signal, receive a second user input responsive to the second candidate transcript, modify the second candidate transcript based on the second user input to generate the second transcript. The first transcription model includes a first machine learning model, and the processor-executable instructions, when executed, further configure the processor to: update the first machine learning model based on the first user input. To determine the third transcript based on the first and second transcripts by using the large language model includes to generate a first candidate transcript using the first transcription model based on the audio signal, receive a first user input responsive to the first candidate transcript, and modify the first candidate transcript based on the first user input to generate the first transcript. To operate the record-keeping application using a desktop automation tool based on the data to generate the patient record includes to store the patient record in a database.

There is disclosed a non-transitory computer-readable medium having stored thereon machine interpretable instructions which. The non-transitory computer readable medium also includes receiving an audio signal indicative of the vocalized expression; generating a first transcript using a first transcription model based on the audio signal; generating a second transcript using a second transcription model based on the audio signal, independently of the first transcription model; determining a third transcript based on the first and second transcripts by using a large language model; using the third transcript and a machine learning model to determine data suitable for generating the patient record; and operating the record-keeping application using a desktop automation tool based on the data to generate the patient record.

Implementations may include one or more of the following features. The non-transitory computer-readable medium where determining the third transcript based on the first and second transcripts by using a large language model includes determining the third transcript based on the first and second transcripts by using a plurality of independent large language models. Determining the third transcript based on the first and second transcripts by using a plurality of independent large language models includes generating a plurality of candidate transcripts using the plurality of independent large language models, each of the plurality of candidate transcripts being uniquely associated with a corresponding one of the plurality of independent large language models; and generating the third transcript based on the plurality of candidate transcripts. Generating the third transcript based on the plurality of candidate transcripts includes receiving a user input responsive to the plurality of candidate transcripts, and modifying the plurality of candidate transcripts based on the user input to generate the third transcript. Generating the third transcript based on the plurality of candidate transcripts includes using a supervised learning model to process the plurality of candidate transcripts to generate the third transcript, and the computer-implemented method further may include: updating the supervised learning model based on the user input. Generating the third transcript based on the plurality of candidate transcripts includes using a supervised learning model to process the plurality of candidate transcripts to generate the third transcript. The large language model is a first large language model, and the machine learning model is a second large language model independent of the first large language model. Generating the first transcript using the first transcription model based on the audio signal includes generating a first candidate transcript using the first transcription model based on the audio signal, receiving a first user input responsive to the first candidate transcript, and modifying the first candidate transcript based on the first user input to generate the first transcript; and generating the second transcript using the second transcription model based on the audio signal, independently of the first transcription model, includes generating a second candidate transcript using the second transcription model based on the audio signal, receiving a second user input responsive to the second candidate transcript, modifying the second candidate transcript based on the second user input to generate the second transcript. The first transcription model includes a first machine learning model, and the computer-implemented method further may include: updating the first machine learning model based on the first user input. Determining the third transcript based on the first and second transcripts by using the large language model includes generating a first candidate transcript using the first transcription model based on the audio signal, receiving a first user input responsive to the first candidate transcript, and modifying the first candidate transcript based on the first user input to generate the first transcript. Operating the record-keeping application using a desktop automation tool based on the data to generate the patient record includes storing the patient record in a database.

In some aspects, there is described a computer-implemented method of automatically generating a record, in a user-oriented record-keeping application, based on vocalized expression. The computer-implemented method includes receiving an audio signal indicative of the vocalized expression; generating a first transcript using a first transcription model based on the audio signal; generating a second transcript using a second transcription model based on the audio signal, independently of the first transcription model; determining a third transcript based on the first and second transcripts by using a plurality of independent large language models; using the third transcript and a machine learning model to determine data suitable for generating the record; and operating the record-keeping application using a desktop automation tool based on the data to generate the record.

In some aspects, there is described a system for automatically generating a record, in a user-oriented record-keeping application, based on vocalized expression. The system includes a processor; and computer-readable memory coupled to the processor and storing processor-executable instructions that, when executed, configure the processor to: receive an audio signal indicative of the vocalized expression, generate a first transcript using a first transcription model based on the audio signal, generate second transcript using a second transcription model based on the audio signal, independently of the first transcription model, determine a third transcript based on the first and second transcripts by using a plurality of independent large language models, use the third transcript and a machine learning model to determine data suitable for generating the record, and operate the record-keeping application using a desktop automation tool based on the data to generate the record.

In some aspects, there is described a non-transitory computer-readable medium. The medium has stored thereon machine interpretable instructions which, when executed by a processor, cause the processor to perform a computer-implemented method of automatically generating a record, in a user-oriented record-keeping application, based on vocalized expression, the computer-implemented method comprising: receiving an audio signal indicative of the vocalized expression; generating a first transcript using a first transcription model based on the audio signal; generating a second transcript using a second transcription model based on the audio signal, independently of the first transcription model; determining a third transcript based on the first and second transcripts by using a plurality of independent large language models; using the third transcript and a machine learning model to determine data suitable for generating the record; and operating the record-keeping application using a desktop automation tool based on the data to generate the record.

Embodiments can include combinations of the above features.

Further details of these and other aspects of the subject matter of this application will be apparent from the detailed description included below and the drawings.

### DESCRIPTION OF THE DRAWINGS

Reference is now made to the accompanying drawings, in which:

FIG. 1 is a schematic of a system for automatically generating a record, in a user-oriented record-keeping application, based on vocalized expression;

FIG. 2 is a schematic block diagram of the system for generating a record, in accordance with an embodiment;

FIG. 3 is a schematic block diagram of the system 100 for generating a record, in accordance with another embodiment;

FIG. 4 is a schematic block diagram of the system 100 for generating a record, in accordance with yet another embodiment;

FIG. 5 is a schematic block diagram of the system 100 incorporating a learning framework, in accordance with an embodiment;

FIG. 6 is an example screenshot of a graphical user interface of the system 100 in a first stage of operation, in accordance with an embodiment;

FIG. 7 is an example screenshot of a graphical user interface of the system 100 in a second stage of operation, in accordance with an embodiment;

FIG. 8 is an example screenshot of a graphical user interface of the system 100 in a third stage of operation, in accordance with an embodiment;

FIG. 9A is an example view of a record-keeping software, in accordance with an embodiment;

FIG. 9B is the example view of the record-keeping software, with data filled therein by the desktop automation tool, in accordance with an embodiment;

FIG. 10 is another example view of the record-keeping software showing an odontogram specification page, in accordance with an embodiment;

FIG. 11 is a schematic flow chart of an exemplary computer-implemented method of automatically generating a record, in a user-oriented record-keeping application, based on vocalized expression;

FIG. 12 illustrates a block diagram of a computing device, in accordance with an embodiment of the present application; and

FIG. 13 is yet another example view of a record-keeping software showing an odontogram specification page, in accordance with an embodiment.

### DETAILED DESCRIPTION

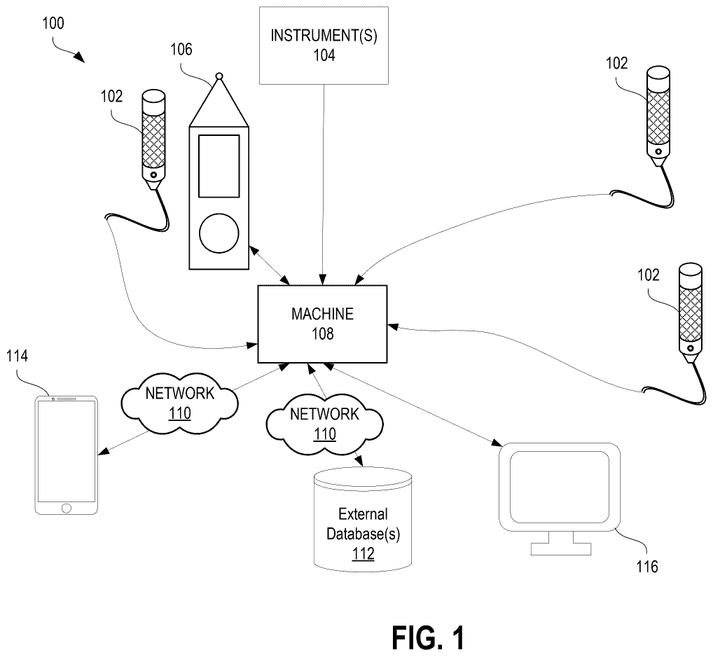

FIG. 1 is a schematic of a system 100 for automatically generating a record, in a user-oriented record-keeping application, based on vocalized expression.

As referred to herein, user-oriented record-keeping applications refers to record-keeping applications, e.g. those incorporating databases or other data structures, that are configured to be operated directly by a user. Many such applications are considered legacy software and/or have been widely adopted by health care providers. Legacy application typically do not have sufficiently capable programmatic interfaces, e.g. APIs, to be operated by other machines to generate records. As such automating, completely or even partially, the generation of records using applications is challenging, which presents an obstacle to improve efficiency in the delivery of health services.

The system 100 may be placed at least partially in the vicinity of individuals so as to allow one or more microphones of the system 100 to capture vocalized expressions of the individuals. It is conceived that the system 100 may be typically positioned so that the one or more microphones are within a physician's examination room, wherein dialogue(s) between patient(s) and doctor(s) may be captured. Nevertheless, it is understood that the technology may be used to capture other types of conversations, e.g. those between other professionals and their clients.

In some embodiments, two or more spatially-separated microphones may be provided so that the sound from a patient (and/or health professional) may be received differently from different microphones. For example, intensity of audio may be indicative of proximity, which may help in distinguishing between speakers in a conversation.

Additionally, instruments 104 may be provided. The instruments may include medical diagnosis tools.

In an exemplary embodiment, the instruments 104 may include tip sensors, body sensors, and/or end sensors. For example, in a dental application, tip sensors may include pressure sensors to measure the firmness of gum tissue and detect odontogram measurements for gum depth. For example, in a dental application, tip sensors may include temperature sensors to detect signs of inflammation or infection. For example, in a dental application, tip sensors may include pH Sensors to assess saliva acidity, indicating potential dental decay or gum disease. For example, in a dental application, body sensors may include high-resolution micro cameras for capturing images of teeth and gums. For example, in a dental application, body sensors may include light sensors, such as LEDs and photodetectors for checking plaque levels and gum coloration. For example, in a dental application, end sensors may include an integrated microphone integrated into the end of a pen to track ambient voice to text measurements by dentists.

In various embodiments, a hardware tool 106 may be provided for dental applications. The hardware tool may have cylindrical shape, similar to a thick pen, and may be ergonomically designed for easy handling and insertion into an oral cavity. The hardware tool may comprise a medical-grade silicone exterior tip for comfort and hygiene, with a machined aluminum body for sterilisation and durability. In various embodiments, the size of the hardware tool 106 may be approximately 15 cm in length and 2.5 cm in diameter, tapering at the end for easy oral insertion.

The hardware tool may comprise one or more sensors mentioned above or otherwise useful in dental applications. The hardware tool may comprise a screen to display information to a user, including information received via a network, wired, or wireless connection. For example, the integrated screen may display odontogram measurements as secondary “double check” helping dentists confirm the system is working properly in real time.

A graphical user interface 116 is provided to display information from a user and allow interaction therewith. For example, the graphical user interface 116 may display processed information for a user to consider. The graphical user interface 116 provides a means for the user to interact with the information and/or indicate a user command or desire. In some embodiments, a display and/or wearable glasses with heads-up display may be provided instead or in addition to the graphical user interface 116.

The microphone(s) 102, instrument(s) 104, hardware tool 106, and/or graphical user interface 116 (and/or display) are communicatively and/or operably connected to a machine 108. The machine 108 may include a processor, memory, and other computing elements useful to generate electronic patient records based on received input. In various embodiments, the machine 108 may at least partially host or have installed thereon an enterprise resource planning (ERP) software for the health professional.

The microphone(s) 102, instrument(s) 104, hardware tool 106, and/or graphical user interface 116 (and/or display or wearable glasses with heads-up display) may be connected to the machine 108 via wired, wireless, or network-based connections. For example, in some embodiments, data may be transmitted to (and from) the machine 108 and the aforementioned devices via WiFi or Bluetooth wireless connections.

In various embodiments, the various devices may be battery-powered.

The machine 108 may be network-connected to a smartphone device 114, external database(s) 112, server(s), and other types of computing systems.

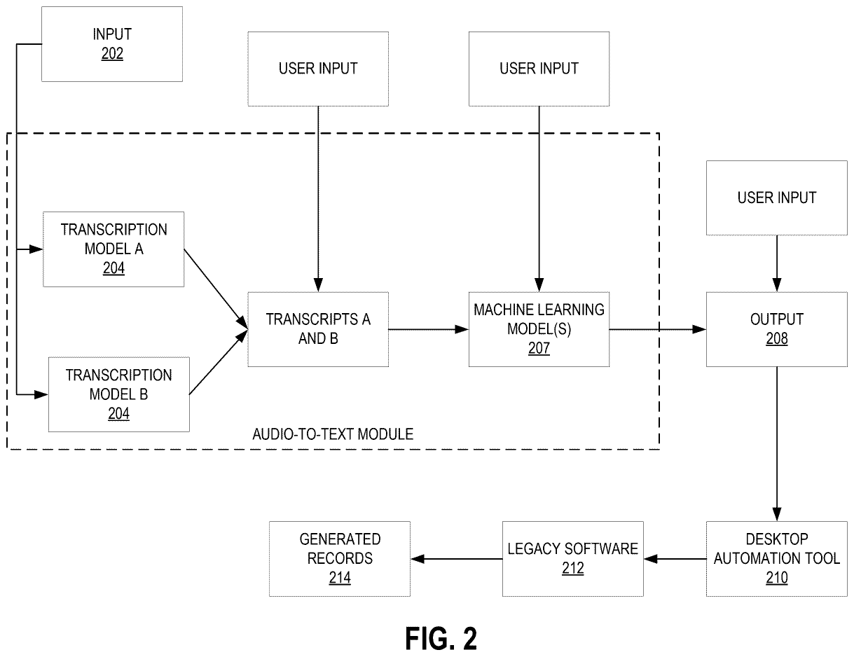

FIG. 2 is a schematic block diagram of the system 100 for generating a record, in accordance with an embodiment.

As shown in FIG. 2, input 202 in the form of audio signal(s) and/or other types of inputs (e.g. data generated by instruments) is provided to two separate, independent transcription models. The audio signals are indicative of vocalized expressions of patient(s) and/or attending professional(s).

The transcription models may be based on classical speech-to-text algorithms or may be machine learning models trained to generate a transcript based on input audio signal(s). In particular, the transcription models A, B are configured to identify vocalized expressions in the audio and form written representations of these. The transcription models may further identify speakers from the audio signal. In some embodiments, the transcription model may identify speakers of various expressions differently depending on the microphone used to capture the vocalized expressions. For example, a plurality of transcripts may be generated by providing the input to the transcription models. In some embodiments, the transcripts may differ from one another, including on important/critical aspects of the patient (patient-professional interaction).

The transcription models A and B may be cloud-based models, e.g. accessible via a network connection and an application programming interface (API). For example, three transcription models known in the art are speech_to_text from Corner Software™ (csdcorp), ElevenLabs™ and WhisperAI™.

State-of-the-art algorithms and technologies for speaker diarization, ensuring accurate identification and separation of speakers in the conversation and handling overlapping speech may be implemented, e.g. End-to-End Neural Diarization (EEND), deep speaker embeddings, self-supervised learning, and/or attention mechanisms.

In some embodiments, one or more of the transcription models may be configured to break down audio into smaller pieces, or to discretize the audio into batches, analyze the pieces or batches, and then decipher the speech by predicting the most likely transcription.

In some embodiments, one or more of the transcription models may be configured to utilize advanced artificial intelligence (AI) and deep learning models to convert spoken language into written text. The system is based on neural networks that are trained to recognize and transcribe human speech in a highly accurate and natural manner.

In some embodiments, one or more of the transcription models may be configured to utilize the built-in speech recognition functionalities provided by the operating system, e.g. Google™ Speech API for Android and Apple™ SFSpeechRecognizer for IOS™.

The plurality of transcripts include a transcript A and a transcript B, generated independently by respective transcription models A and B based on the input, e.g. audio signal therein.

The transcripts A, B may then be viewable and/or editable by a user. For example, the transcripts A, B may be editable during a learning phase of the system and may be only viewable thereafter. The user may edit the transcripts A, B to remove errors or make adjustments, as appropriate.

The transcripts A, B are then used by one or more machine learning models to generate an output 208 in the form of data suitable for generating a patient record. For example, the output may include a summary of the patient interaction and structured text representing information gleaned from the interaction or determined during the interaction.

The machine learning model may be configured to identify a number and identity of speakers, distinguish between speakers, and determine patient input as well as a dentist's questions and diagnoses. The machine learning model may determine output suitable for entry into the legacy software 212 based on transcripts, audio signals, the location and/or microphone where the audio signals (and associated transcripts) originate from, and instrument inputs. For example, output of an image sensor may be used to specify diagnoses in addition to vocally expressed diagnoses by the dentist that are reflected in one or more of the transcripts. For example, a measured sensitivity captured by an instrument connected to the machine 108 may be used in addition to a vocally expressed sensitivity that is reflected in one or more of the transcripts.

In various embodiments, the machine learning model may include supervised machine learning models, unsupervised machine learning models, generative machine learning models, and/or large language models.

A desktop automation tool is then used to operate the record-keeping application (see legacy software 212). The desktop automation tool may conduct such operation based on the output 208 (the data) so as to enter the data into the record-keeping application without further user intervention. The desktop automation tool may thereby facilitate generation of a record 214. Advantageously, in various embodiments, health professionals' practices may be made more efficient while mitigating disruption to existing systems and processes of health professionals.

As referred to herein, desktop automation tools may generally refer to tools that allow operation of a user-interface via pre-programmed actions or predetermined behaviours. For example, desktop automation tools may allow automatic or predetermined operation of a user's mouse cursor to position said mouse cursor over a field over a form, and then cause a selection (via the mouse cursor) of the field to cause activation thereof. The desktop automation tool may then emulate the user's keystrokes, or data entry, to fill the field based on the data.

Desktop automation tools may include cloud-based automation. For example, desktop automation tools may be operated via an API or may otherwise be API-accessible. Advantageously, this may improve platform agnosticism of technologies presented herein. An example of a desktop automation tool is nutjs™.

In some embodiments, an application programming interface (API) may be used to enter the data into a record-keeping application.

The system components used to generate output 208 (the data used by the desktop automation tool) may be generally referred to collectively as the audio-to-text module.

In various embodiments, the audio-to-text module may generate speaker profiles for recurring speakers (e.g., dentists, hygienists), which may improve identification accuracy over time. For example, the speaker profiles may be used in the transcription models and/or the LLMs (or other machine learning models).

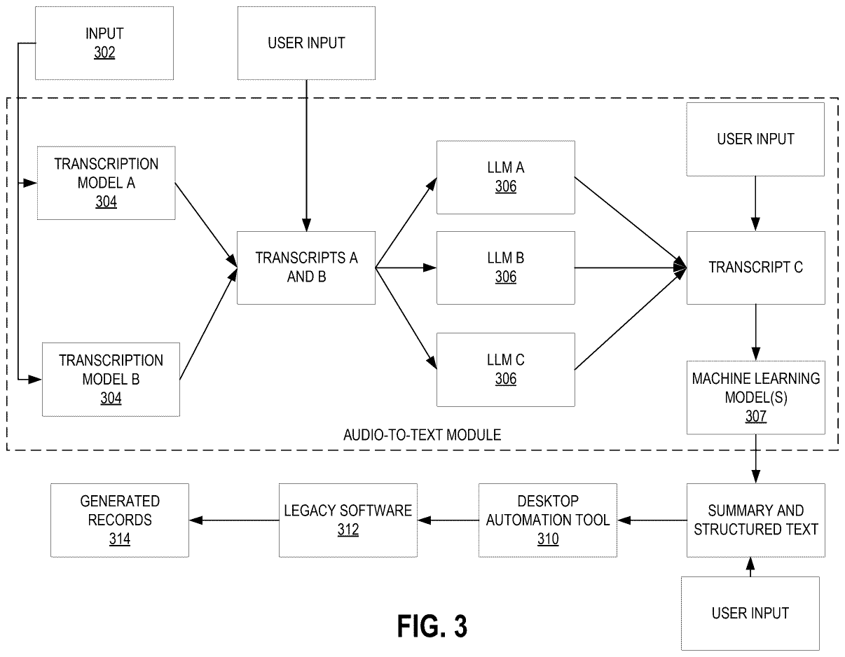

FIG. 3 is a schematic block diagram of the system 100 for generating a record, in accordance with another embodiment.

In the embodiment shown in FIG. 3, the transcripts A, B are used by one or more machine learning models to generate a transcript C, which may be a result of combining or harmonizing transcript A and transcript B. In particular, the transcript C is determined based on the transcript A and transcript B by using a plurality of independent large language models (LLMs), e.g. LLM A 306, LLM B 306, LLM C 306. Each of these models may be queried to generate a candidate transcript from the two transcripts A and B. The resulting plurality of candidate transcripts may then be provided to one or more of the same large language models to determine the transcript C. Each LLM may be defined at least in part by its weights, which form the LLM's weight set. In various embodiments, each of the plurality of independent large language models is defined by a corresponding weight set of a plurality of weight sets that are distinct from each other.

The LLMs may be accessible by APIs and/or may be cloud-based. For example, a first LLM may be one of OpenAI™'s ChatGPT™ models, a second LLM may be one of Anthropic™'s Claude™ models, and a third LLM may be one of Meta™'s LLaMA™ family of open-source models, a fourth LLM may be xAI™'s Grok™ model, and so on. The LLMs may be generic LLMs. For example, LLMs may be trained on trained general internet data. Examples of datasets for training LLMS include Common Crawl™, RefinedWeb™, The Pile™, C4™, Starcoder™, BookCorpus™, ROOTS™, Wikipedia™, and Red Pajama™. Advantageously, diverse model integration may be used, e.g. integration with a wide range of AI models beyond those specified above, such as BERT, T5, ROBERTa, and specialized dental language models. This diversity ensures a comprehensive evaluation from multiple perspectives. For example, five or more LLMs may be used to determine and process transcript(s).

In some embodiments, the LLMs may be custom LLMs, e.g. a BERT (Bidirectional Encoder Representations from Transformers) model trained on authentic or synthetic patient record data.

In some embodiments, the transcript C may be viewable and/or editable by the user. For example, the transcript C may be editable during a learning phase of the system and may be only viewable thereafter. The user may edit the transcript C to remove errors or make adjustments, as appropriate. In some embodiments, a user may provide in put to the one or more machine learning model(s) to generate the third transcript C.

A machine learning model 307 (which may comprise one or more subsidiary machine learning model) may generate data based on the transcript C, the data being suitable for being used with the desktop automation tool 310 to generate records via legacy software. For example, in some embodiments, the machine learning model may comprise one or more LLMs that are queried to extract information required to be input into the legacy software by the desktop automation tool. For example, the one or more LLMs may be the same as or may comprise the plurality of LLMs (LLM A, LLM B, LLM C). In some embodiments, the machine learning model(s) 307 may include an LLM for converting the transcript C into standard form and/or a supervised learning model for extracting relevant information from the transcript C for entering into the legacy software (or user-oriented record-keeping application).

In the embodiment shown in FIG. 3, the one or more machine learning algorithms generates a summary of the transcript C and structured text for use by the desktop automation tool. Examples of structured text formats include JSON and XML text formats.

In the embodiment shown in FIG. 3, a user can provide input in the form of edits to the any one or more of the transcripts A, B, or C. A user may additional provide input to the summary and structured text in order to achieve a desired accuracy of the generated record(s).

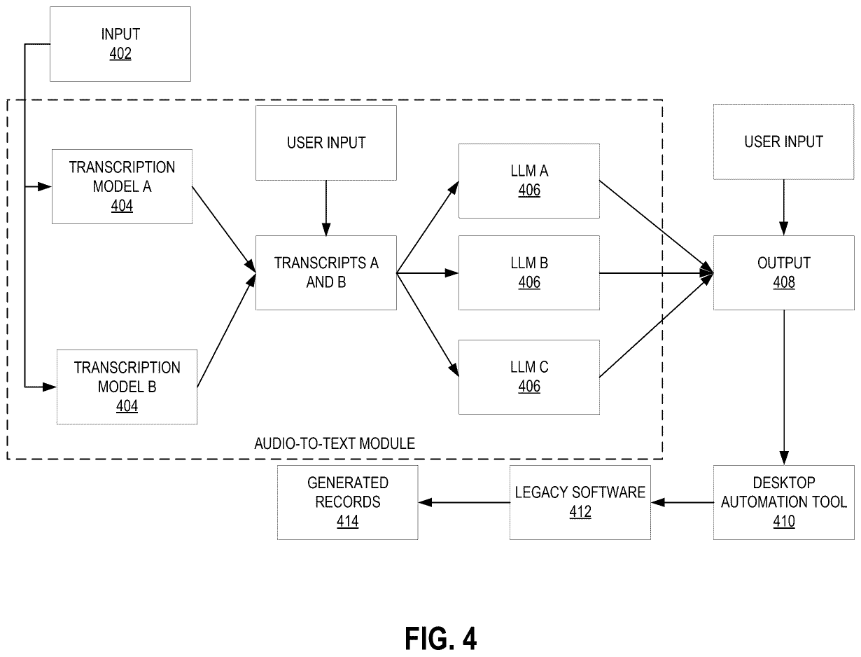

FIG. 4 is a schematic block diagram of the system 100 for generating a record, in accordance with yet another embodiment.

In the embodiment of FIG. 4, the plurality of LLMs use transcripts A and B to directly generate outputs suitable for use with the desktop automation tool in order to operate the user-oriented record-keeping application. The user may provide input in the form of edits to the transcripts A and/or B, as well as in the form of edits to the output 408.

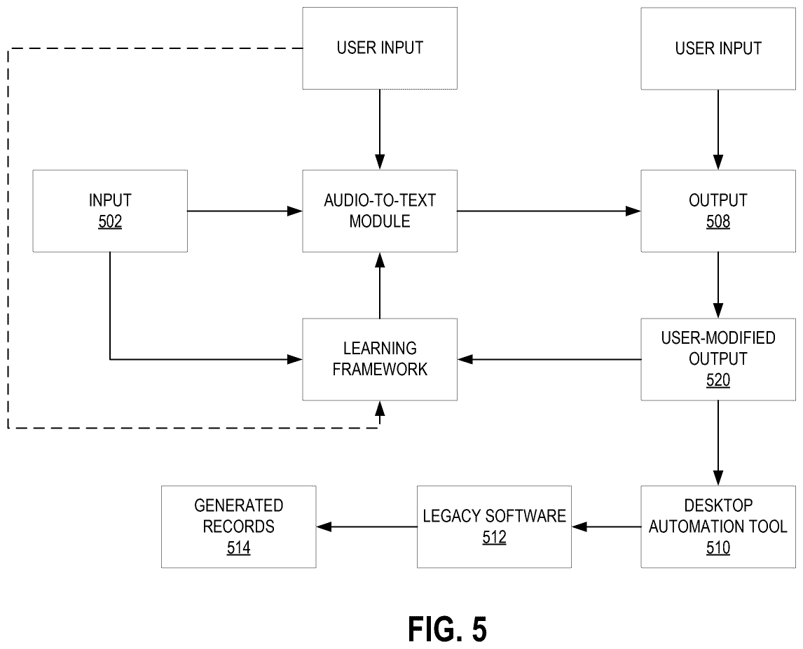

FIG. 5 is a schematic block diagram of the system 100 incorporating a learning framework, in accordance with an embodiment.

In the embodiment of FIG. 5, the audio-to-text module may incorporate one or more machine learning models, e.g. supervised machine learning models, unsupervised machine learning models, generative models, and/or large language models, as described previously.

The embodiment of FIG. 5 incorporates a learning framework, e.g. a module that allows updating of the audio-to-text module and/or machine learning models therein based on learning a user's preferences. In some embodiments, the user-modified output is used in conjunction with the input (to form training data) to further train the audio-to-text module to match a user's preferences and desired objectives. For example, a supervised learning algorithm may be incrementally updated based on such training data (fine-tuned). In some embodiments, user inputs entered in the form of edits to the transcripts are also used by the audio-to-text module to fine tune machine learning models thereof.

In some embodiments, the learning framework may be configured to update the audio-to-text module during a learning phase of the system. For example, the learning phase may be an initial phase of operation of the system. Once the system is sufficiently well-trained, the system may enter into a non-learning phase, wherein the learning framework may be configured to stop updating the audio-to-text module. In various embodiments, the non-learning phase may be triggered by the user and/or by metrics indicative of insufficient marginal change in outputs with further training.

Continuous Model Training using real-time data collection allows the audio-to-text module to focus on dental-specific terminologies and conversational nuances to maintain the relevance and accuracy of the audio-to-text module.

In various embodiments, the learning framework may implement adaptive learning mechanisms that allow AI models to learn from their mistakes and improve over time based on the feedback received from both experts and crowdsourced evaluations. In various embodiments, the learning framework may be configured to implement real-time updates to the AI models that incorporate the latest dental practices, terminologies, and conversational patterns. In various embodiments, the learning framework may facilitate dental professionals to review and validate the AI transcripts, which human oversight may promote the AI models' decisions align with practical expectations and domain expertise.

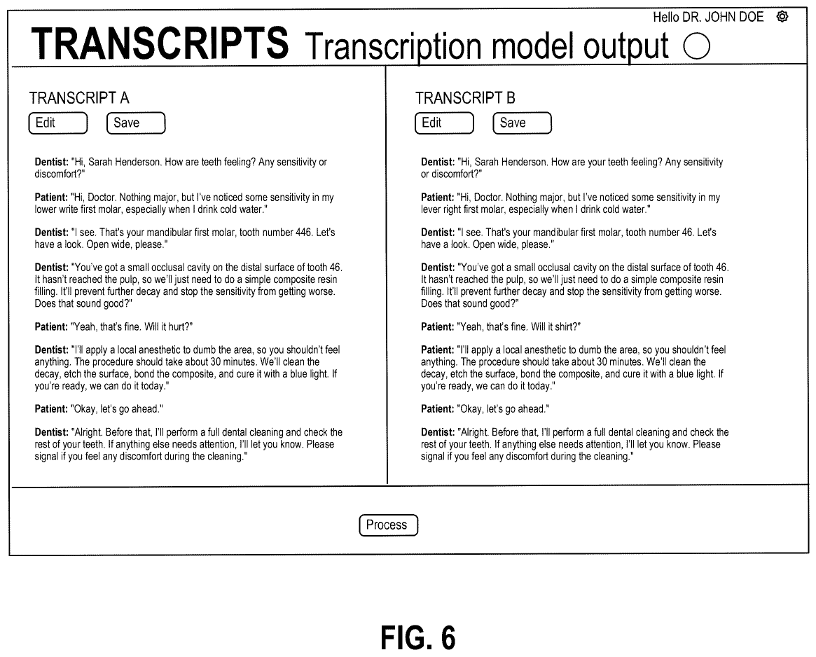

FIG. 6 is an example screenshot of a graphical user interface of the system 100 in a first stage of operation, in accordance with an embodiment.

The screenshot may be shown to a user (e.g. a health professional) after the audio is processed by a plurality (at least two) of transcription models.

The user may be shown a first transcript (Transcript A) and a second transcript (Transcript B). While the two transcripts may be generally similar, they may differ in important ways. For example, in Transcript A, the dentist identifies tooth 446 while in Transcript B, the dentist identifies tooth 46. Similarly, there is disagreement between Transcript A and B as to the speaker of certain portions of the transcript.

With patient engagement extending to up to an hour, such transcripts may become lengthy, and it can become difficult for a user to keep track of and analyze the various transcripts in detail to identify any points of confusion or error. In some embodiments, the user has an option to edit the transcripts before proceeding to the next step. For example, in the screenshot shown, a dentist may immediately identify “tooth 446” as being an incorrect transcription, since there exists no such tooth. As such, a dentist may edit Transcript A to change the reference to tooth 446.

In some embodiments, the user may be able to edit the transcripts only during a learning phase. In some embodiments, the user may be able to begin editing the transcripts by simply selecting the field of choice and entering desired edits.

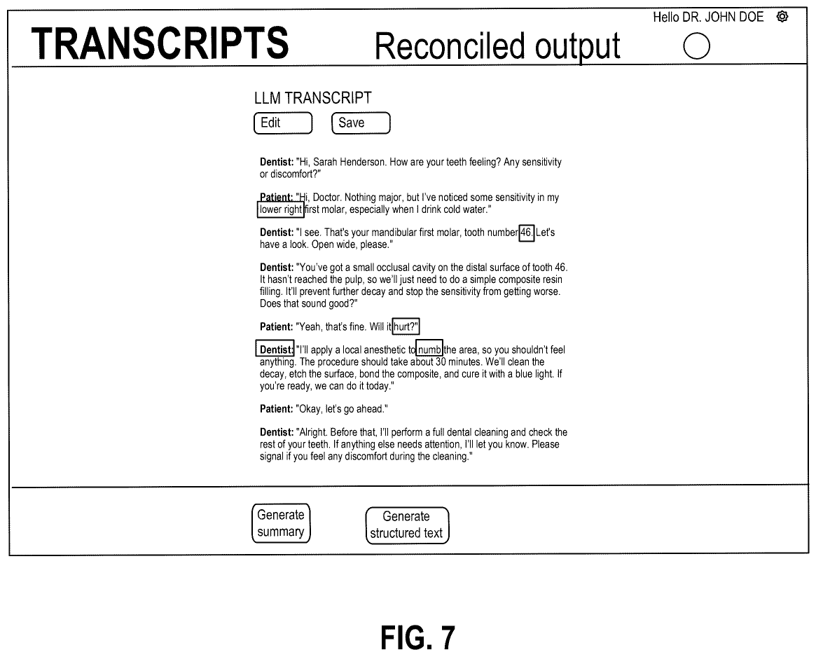

FIG. 7 is an example screenshot of a graphical user interface of the system 100 in a second stage of operation, in accordance with an embodiment.

The transcript shown in FIG. 7 is a transcript achieved by processing transcripts A and B via machine learning model(s), e.g. the plurality of LLMs. In an exemplary embodiment, three general purpose LLMs may be used to determine the transcript. As mentioned previously, such processing may include determining speakers based partially on the outputs from various microphones. The graphical user interface may highlight the locations where the transcripts differed and/or were determined to be inaccurate by the machine learning model(s). For example, in the transcript shown in FIG. 7, the dentist is identified as describing the steps of a procedure.

In some embodiments, a voting mechanism is used. For example, each AI model may evaluate which transcript makes more sense and the transcript that receives approval from at least two out of the three AI models is chosen to be model transcript.

In some embodiments, a voting mechanism may be implemented to select a (preferred) transcript from a plurality of transcripts, which may be generated by one or more (different) transcription models.

A voting mechanism may select the best transcription result from multiple voice-to-text (VTT) systems, and eventually replaces the human expert with a fine-tuned supervised language model (SLM). Examples of VTT systems include ElevenLabs™ Google™ Speech API, Apple™ SFSpeechRecognizer, and Whisper™.

In some embodiments, a first part of the voting mechanism is initial system design for a multimodal voting process. This involves combining all the outputs from the different VTT systems and introducing a human expert and a large language model (LLM) for semantic evaluation. The goal is to assign confidence scores to each transcription and eventually train an SLM to simulate the expert's role.

In some embodiments, step one of the first part is transcription collection. Each VTT system may produce its own transcription. A human expert (user, dentist, or external reviewer) may review and correct any errors in each transcription. A large-language model (LLM) may evaluate the transcriptions based on language coherence, syntax, context, and grammar.

In some embodiments, step two of the first part is confidence scoring. For each transcription, a confidence score may be calculated based on the following parameters: textual similarity, LLM contextual evaluation, and human expert review. Textual similarity may involve comparing each VTT output to the other transcriptions (other VTT outputs) using metrics such as Levenshtein distance or cosine similarity and if the outputs agree (similarity is high), the transcription receives a higher score. LLM contextual evaluation may involve the LLM analyzing grammar, meaning, and fluency, and assignment of an additional score based on the LLM comparing the transcription to typical phrasing. In human expert review, an expert's corrections and decisions may serve as the final arbiter and may determine the highest priority score for that session.

In some embodiments, step three of the first part involves an aggregated voting mechanism and/or weighted voting. Each transcription's final score may combine the confidence score from the VTT system, LLM review, and expert assessment, weighted by reliability. For example, initial weights could be distributed as: 40% weight for VTT outputs (based on initial accuracy testing across systems), 30% for the LLM assessment, and 30% for the human expert's opinion.

The transcription with the highest cumulative score is then selected as the preferred transcription for the session.

In some embodiments, a second part of the voting mechanism is training a Supervised Language Model (SLM) or a supervised learning model, the goal being to eventually train an SLM to replace the human expert.

In some embodiments, step one of the second part involves dataset preparation. The system may be configured to collect labeled datasets that include VTT outputs, the human expert's corrections, and decisions from the LLM-based scoring system. Each transcription session, along with its chosen final output, may become a labeled training example for the SLM. Steps may be taken to ensure that the dataset is large enough to cover diverse scenarios, including different accents, noisy environments, varying speech speeds, and specialized terminology.

In some embodiments, step two of the second part involves model architecture. Transformer-based models such as GPT or BERT, fine-tuned on the task of transcription correction and evaluation, may be used. The model architecture should be designed to take multiple transcription inputs (e.g., from ElevenLabs, Google, Apple, Whisper) and provide a corrected output, similar to how the human expert operates.

In some embodiments, step three of the second part involves model training. The SLM may be trained using the collected dataset with multiple inputs (VTT outputs) and the final transcription chosen by the expert as the target. Error analysis may be incorporated by comparing the expert decisions with VTT system outputs to focus on edge cases where the SLM should learn corrective behaviors.

In some embodiments, step four of the second part involves fine-tuning with expert corrections. As the system is deployed and refined, real-time expert corrections is fed back into the training loop to continuously fine-tune the SLM. Over time, this loop may gradually reduce dependency on human input as the SLM improves.

In some embodiments, step four of the second part involves validation and benchmarking. The SLM's performance may be periodically benchmarked against human expert decisions to validate its performance. Improvements may be tracked and on edge cases where human intervention may still be required may be the subject of focus. Additionally, A/B testing may be used to gradually replace the human expert with the SLM in certain cases and to measure performance.

In some embodiments, a third part of the voting mechanism may involve feedback loop and system evolution. In one aspect of the third part, continuous learning is provided. In another aspect of the third part, weight adjustment is provided. In continuous learning, each transcription session may provide a feedback loop where the human expert's decisions are used to continuously improve both the LLM's evaluation and the SLM's correction capabilities. In weight adjustment, the weights for each component (VTT systems, LLM, human) may be adjusted dynamically as the system learns which VTT system performs best in specific environments or contexts (e.g., noisy vs. quiet settings). As the SLM gets better, the role of the human expert may phased out, allowing the SLM to replace the expert in real-time transcription evaluation and correction. For example, this may occur over a long-term period.

An example workflow may be as follows: (a) speech sample: a speaker provides an n-second audio clip; (b) VTT transcription: ElevenLabs™, Google ™, Apple™, and Whisper™ transcribe the speech, with outputs as follows: ElevenLabs™ “The brown fox jumps over the lazy dog.”, Google™—“The brown fox jump over the lazy dog.”, Apple™—“Brown fox jumped over lazy dog.”, Whisper™—“The fox jumped over the lazy dog.”; (d) LLM evaluation: the LLM flags the Google™ result for incorrect subject-verb agreement and suggests ElevenLabs™ as the best transcription; (e) human expert: the expert confirms that ElevenLabs™' transcription is the most accurate; (f) voting result: based on similarity scores, LLM feedback, and expert judgment, the ElevenLabs™ transcription is selected; and (g) SLM training: this session is used as training data for the SLM, improving its ability to make the human expert redundant in future cases.

By combining these steps, a robust, self-learning system may be created that evolves from relying on multiple VTT systems and human experts to using an SLM that can autonomously handle transcription evaluation over time.

A user may then select whether to generate a summary and/or generate structured text.

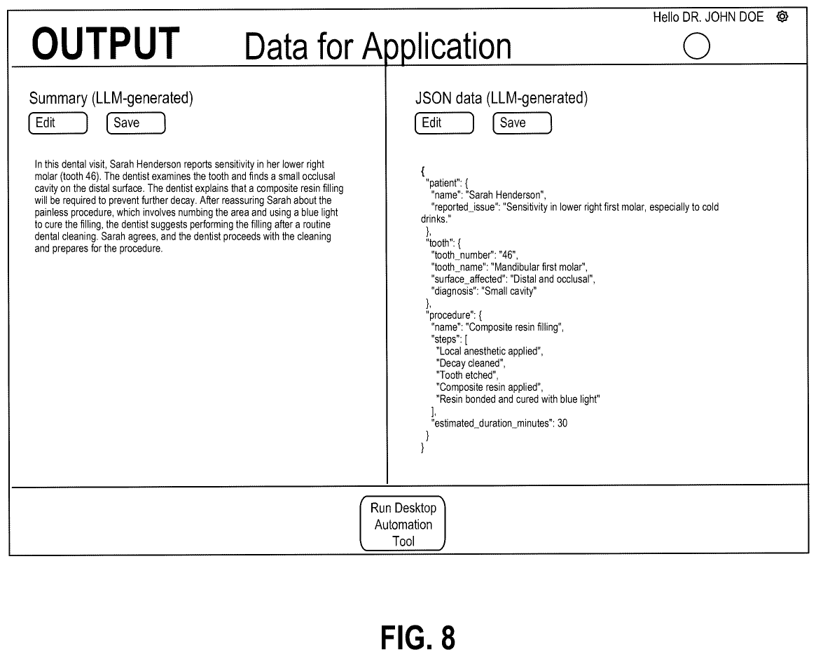

FIG. 8 is an example screenshot of a graphical user interface of the system 100 in a third stage of operation, in accordance with an embodiment.

In the screenshot in FIG. 8, the output data for the record-keeping application is shown. The output data here includes a summary and JSON data. The data may be editable and viewable during a learning phase of the system. The data may only be viewable, and not editable, during a non-learning phase of the system.

In some embodiments, the system may be configured to identify voice-activated input. For example, a dentist may provide voice instructions which may be recognized as instructions by the audio-to-text module, which may then operate the machine 108 accordingly.

FIG. 8 shows example information extract from a transcript. Extracted information may include:
Tooth numbering: universal numbering system (1-32 for permanent teeth, A-T for primary teeth in the USA). Other systems include FDI (Fédération Dentaire Internationale) and Palmer notation.
Surface conditions: condition of each tooth surface (mesial, distal, occlusal, buccal, lingual) such as decay, fillings, crowns, and fractures.
Types of restorations: different types of restorations such as amalgam, composite, gold fillings; porcelain, gold, PFM crowns; bridges and dentures.
Tooth status: status of each tooth, such as present, missing, impacted, extracted, unerupted.
Periodontal information: details such as pocket depths, gingival recession, and bone levels.
Endodontic treatments: treatments such as root canals, pulpotomy, and apicoectomy.
Prosthetics: information on fixed prosthetics like crowns and bridges, and removable prosthetics like partial and full dentures.
Orthodontic information: details such as brackets, bands, wires, and/or space maintainers.
Conditions and observations, e.g. observations on caries (cavities), attrition, abrasion, erosion, and/or hypoplasia.

Additional annotations may include notes on soft tissues, any pathology, and/or special conditions like supernumerary teeth.

In some embodiments, the information extracted from the transcript and, in particular, treatment information extracted from the transcript may be used to determine treatment steps taken and/or treatment costs incurred as a result. For example, the system may be configured to determine or infer a code, e.g. an insurance code, a treatment code, or a diagnosis code, based on the data. For example, insurance codes in health insurance are standardized numerical or alphanumeric codes used to identify specific medical procedures, diagnoses, treatments, and services for billing and claims purposes. Examples of codes include: CPT™ Codes (Current Procedural Terminology), used for medical services and procedures; HCPCS Codes (Healthcare Common Procedure Coding System), used for services, equipment, and supplies not covered by CPT codes; and ICD Codes (International Classification of Diseases), used to identify diagnoses. For examples, codes may facilitate consistency and accuracy in billing between healthcare providers and insurance companies. In some embodiments, advantageously, direct automated billing from patient conversations may thereby be achieved.

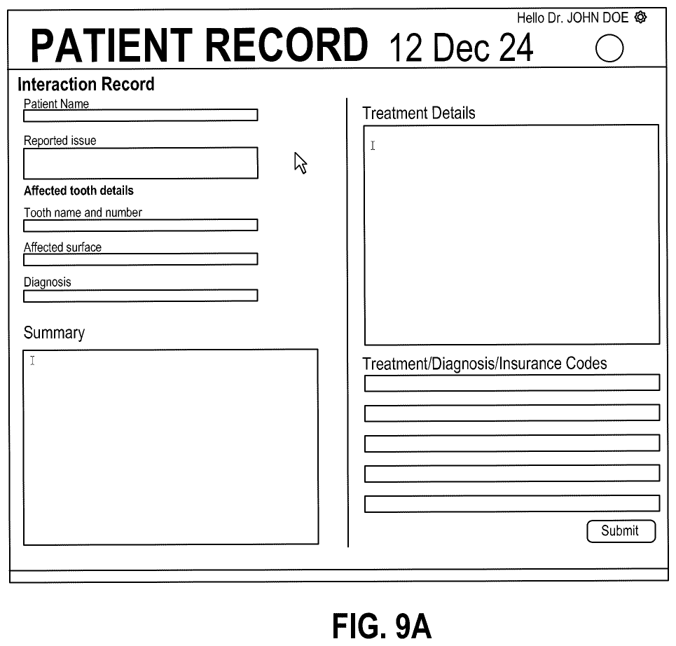

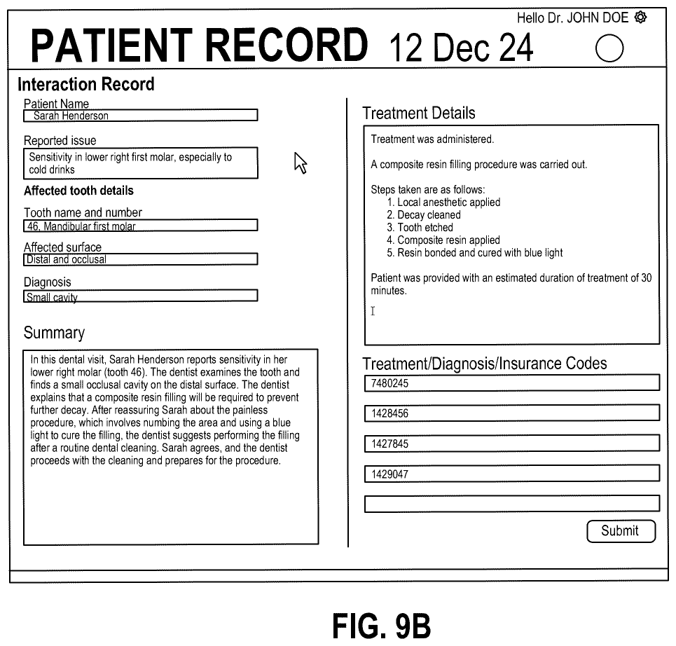

FIG. 9A is an example view of a record-keeping software, in accordance with an embodiment.

FIG. 9B is the example view of the record-keeping software, with data filled therein by the desktop automation tool, in accordance with an embodiment.

The data in FIG. 8 is shown applied and enter into the application. In various embodiments, the data is used to generate narrative text, which is then entered into the application.

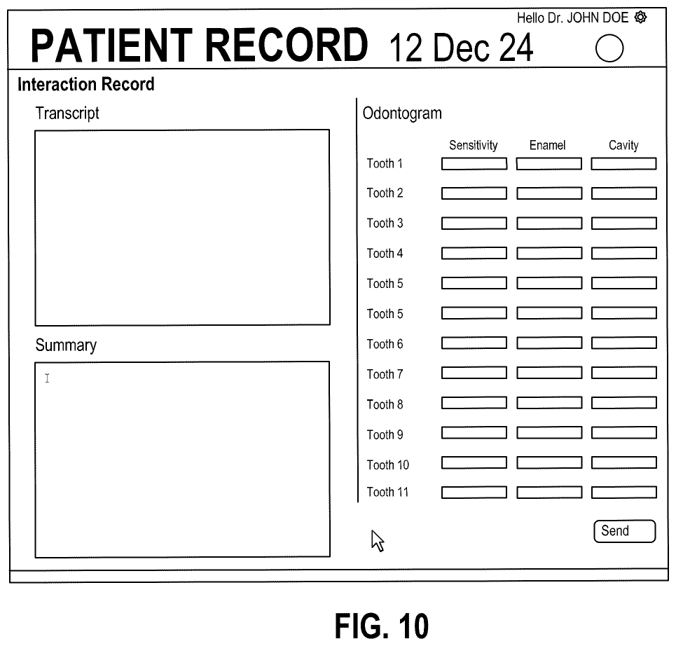

FIG. 10 is another example view of the record-keeping software showing an odontogram specification page, in accordance with an embodiment. Such a specification page may be filled out via methods and systems disclosed herein.

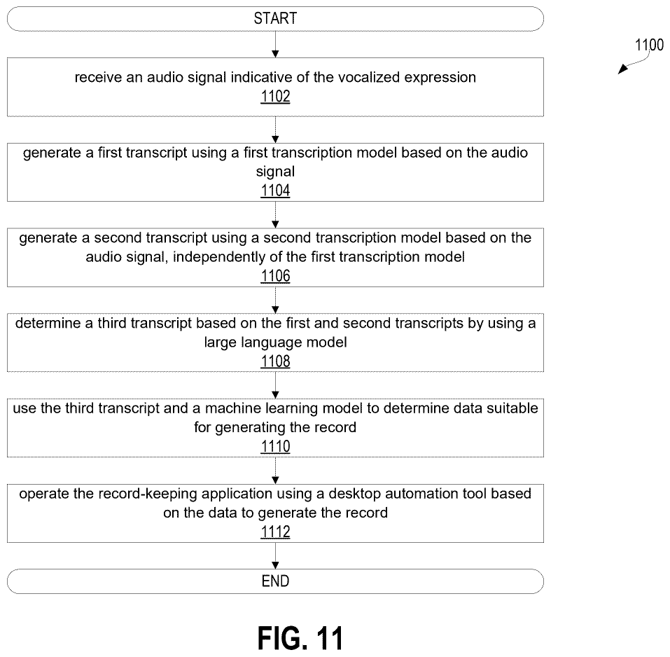

FIG. 11 is a schematic flow chart of an exemplary computer-implemented method 1100 of automatically generating a patient record, via a user-oriented record-keeping application, based on vocalized expression.

Step 1102 of the method 1100 includes receiving an audio signal indicative of the vocalized expression.

Step 1104 of the method 1100 includes generating a first transcript using a first transcription model based on the audio signal.

Step 1106 of the method 1100 includes generating a second transcript using a second transcription model based on the audio signal, independently of the first transcription model.

Step 1108 of the method 1100 includes determining a third transcript based on the first and second transcripts by using a plurality of independent large language models.

Step 1110 of the method 1100 includes using the third transcript and a machine learning model to determine data suitable for generating the record.

Step 1112 of the method 1100 includes operating the record-keeping application using a desktop automation tool based on the data to generate the record.

As referred to here, an audio signal may include data indicative of the vocalized expression.

In some embodiments of the method 1100, determining the third transcript based on the first and second transcripts by using a large language model includes determining the third transcript based on the first and second transcripts by using a plurality of independent large language models.

In some embodiments of the method 1100, determining the third transcript based on the first and second transcripts by using a plurality of independent large language models includes generating a plurality of candidate transcripts using the plurality of independent large language models, each of the plurality of candidate transcripts being uniquely associated with a corresponding one of the plurality of independent large language models; and generating the third transcript based on the plurality of candidate transcripts.

In some embodiments of the method 1100, generating the third transcript based on the plurality of candidate transcripts includes receiving a user input responsive to the plurality of candidate transcripts, and modifying the plurality of candidate transcripts based on the user input to generate the third transcript. For example, the plurality of candidate transcripts may generated by LLMs, and a user may interact with such transcripts to provide adjustments and corrections.

In some embodiments of the method 1100, generating the third transcript based on the plurality of candidate transcripts includes using a supervised learning model to process the plurality of candidate transcripts to generate the third transcript, the method further comprising updating the supervised learning model based on the user input. For example, advantageously, the preferences and proclivities of professional users may thereby be encoded into a machine learning framework.

In some embodiments of the method 1100, generating the third transcript based on the plurality of candidate transcripts includes using a supervised learning model to process the plurality of candidate transcripts to generate the third transcript.

In some embodiments of the method 1100, the large language model is a first large language model, and the machine learning model is a second large language model independent of the first large language model.

In some embodiments of the method 1100, generating the first transcript using the first transcription model based on the audio signal includes generating a first candidate transcript using the first transcription model based on the audio signal, receiving a first user input responsive to the first candidate transcript, and modifying the first candidate transcript based on the first user input to generate the first transcript; and generating the second transcript using the second transcription model based on the audio signal, independently of the first transcription model, includes generating a second candidate transcript using the second transcription model based on the audio signal, receiving a second user input responsive to the second candidate transcript, and modifying the second candidate transcript based on the second user input to generate the second transcript.

In some embodiments of the method 1100, the first transcription model includes a first machine learning model, the method further comprising: updating the first machine learning model based on the first user input. For example, advantageously, real-time learning of user preferences and characteristics may be achieved.

In some embodiments of the method 1100, determining the third transcript based on the first and second transcripts by using the large language model includes generating a first candidate transcript using the first transcription model based on the audio signal, receiving a first user input responsive to the first candidate transcript, and modifying the first candidate transcript based on the first user input to generate the first transcript.

In some embodiments of the method 1100, operating the record-keeping application using a desktop automation tool based on the data to generate the patient record includes storing the patient record in a database. For example, a physical change in a server storage system may occur as result.

In some embodiments, there is provided a non-transitory computer-readable medium having stored thereon machine interpretable instructions which, when executed by a processor, cause the processor to perform a computer-implemented method of automatically generating a record, in a user-oriented record-keeping application, based on vocalized expression, the computer-implemented method 1100.

In some embodiments, there is provided a system for automatically generating a record, in a user-oriented record-keeping application, based on vocalized expression, comprising: a processor; and computer-readable memory coupled to the processor and storing processor-executable instructions that, when executed, configure the processor to execute the method 1100.

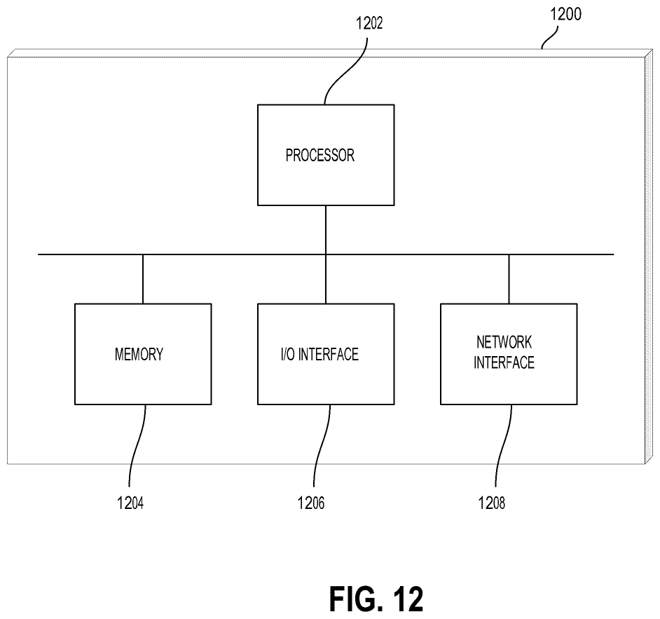

FIG. 12 illustrates a block diagram of a computing device 1200, in accordance with an embodiment of the present application.

As an example, one or more parts of the system 100, one or more parts of the instrument(s) 104, the machine 108, the smartphone 114, one or more servers, the audio-to-text module, the desktop automation tool, the legacy software (user-oriented application) may be implemented using the example computing device 1200 of FIG. 12. The method 1100 may further be implemented using the computing device 1200.

The computing device 1200 includes at least one processor 1202, memory 1204, at least one I/O interface 1206, and at least one network communication interface 1208.

The processor 1202 may be a microprocessor or microcontroller, a digital signal processing (DSP) processor, an integrated circuit, a field programmable gate array (FPGA), a reconfigurable processor, or combinations thereof.

The memory 1204 may include a computer memory that is located either internally or externally such as, for example, random-access memory (RAM), read-only memory (ROM), compact disc read-only memory (CDROM), electro-optical memory, magneto-optical memory, programmable read-only memory (PROM), erasable programmable read-only memory (EPROM), and electrically-erasable programmable read-only memory (EEPROM), Ferroelectric RAM (FRAM).

The I/O interface 1206 may enable the computing device 1200 to interconnect with one or more input devices, such as a keyboard, mouse, camera, touch screen and a microphone, or with one or more output devices such as a display screen and a speaker.

The networking interface 1208 may be configured to receive and transmit data sets representative of the machine learning models, for example, to a target data storage or data structures. The target data storage or data structure may, in some embodiments, reside on a computing device or system such as a mobile device.

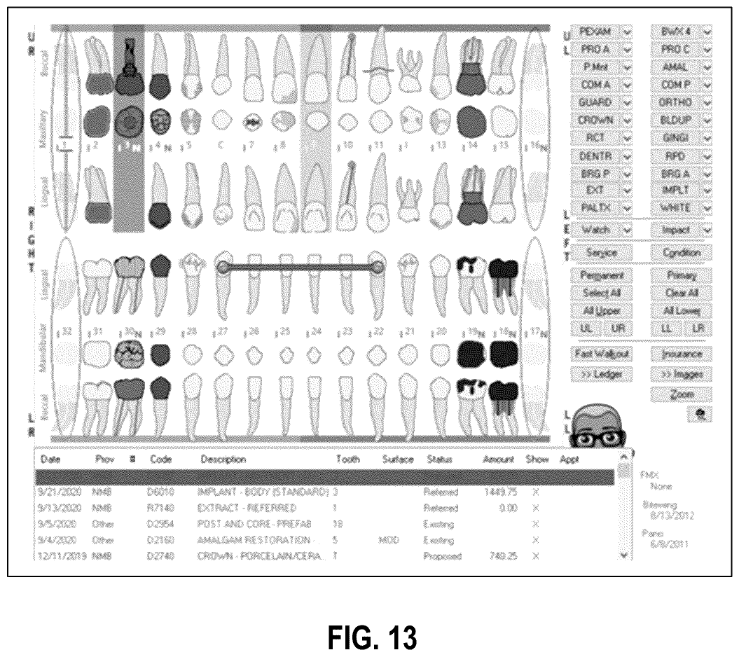

FIG. 13 is yet another example view of a record-keeping software showing an odontogram specification page, in accordance with an embodiment.

In the example shown in FIG. 13, each tooth may be schematically depicted and notes may be entered describing each of the teeth. For example, selecting a tooth may activate (or focus) a tooth-specific note or a tooth-specific input interface to allow notes to be entered that are specific to the tooth of interest.

The embodiments described in this document provide non-limiting examples of possible implementations of the present technology. Upon review of the present disclosure, a person of ordinary skill in the art will recognize that changes may be made to the embodiments described herein without departing from the scope of the present technology. For example, aspects disclosed herein may be used in non-dental health settings, and in non-health settings. Yet further modifications could be implemented by a person of ordinary skill in the art in view of the present disclosure, which modifications would be within the scope of the present technology.

Although the embodiments have been described in detail, it should be understood that various changes, substitutions and alterations can be made herein without departing from the scope. Moreover, the scope of the present application is not intended to be limited to the particular embodiments of the process, machine, manufacture, composition of matter, means, methods and steps described in the specification.

What is claimed is:

1. A computer-implemented method of automatically generating a patient record, via a user-oriented record-keeping application, based on vocalized expression, comprising:
receiving an audio signal indicative of the vocalized expression;
generating a first transcript using a first transcription model based on the audio signal;
generating a second transcript using a second transcription model based on the audio signal, independently of the first transcription model;
determining a third transcript based on the first and second transcripts by using a large language model;
using the third transcript and a machine learning model to determine data suitable for generating the patient record; and
operating the record-keeping application using a desktop automation tool based on the data to generate the patient record.

2. The method of claim 1, wherein determining the third transcript based on the first and second transcripts by using a large language model includes determining the third transcript based on the first and second transcripts by using a plurality of independent large language models.

3. The method of claim 2, wherein determining the third transcript based on the first and second transcripts by using a plurality of independent large language models includes
generating a plurality of candidate transcripts using the plurality of independent large language models, each of the plurality of candidate transcripts being uniquely associated with a corresponding one of the plurality of independent large language models; and
generating the third transcript based on the plurality of candidate transcripts.

4. The method of claim 3, wherein generating the third transcript based on the plurality of candidate transcripts includes
receiving a user input responsive to the plurality of candidate transcripts, and
modifying the plurality of candidate transcripts based on the user input to generate the third transcript.

5. The method of claim 4, wherein generating the third transcript based on the plurality of candidate transcripts includes using a supervised learning model to process the plurality of candidate transcripts to generate the third transcript, the method further comprising:
updating the supervised learning model based on the user input.

6. The method of claim 3, wherein generating the third transcript based on the plurality of candidate transcripts includes using a supervised learning model to process the plurality of candidate transcripts to generate the third transcript.

7. The method of claim 1, wherein the large language model is a first large language model, and the machine learning model is a second large language model independent of the first large language model.

8. The method of claim 1, wherein
generating the first transcript using the first transcription model based on the audio signal includes
generating a first candidate transcript using the first transcription model based on the audio signal,
receiving a first user input responsive to the first candidate transcript, and
modifying the first candidate transcript based on the first user input to generate the first transcript; and
generating the second transcript using the second transcription model based on the audio signal, independently of the first transcription model, includes
generating a second candidate transcript using the second transcription model based on the audio signal,
receiving a second user input responsive to the second candidate transcript, and
modifying the second candidate transcript based on the second user input to generate the second transcript.

9. The method of claim 8, wherein the first transcription model includes a first machine learning model, the method further comprising:
updating the first machine learning model based on the first user input.

10. The method of claim 8, wherein
determining the third transcript based on the first and second transcripts by using the large language model includes
generating a first candidate transcript using the first transcription model based on the audio signal,
receiving a first user input responsive to the first candidate transcript, and
modifying the first candidate transcript based on the first user input to generate the first transcript.

11. The method of claim 1, wherein operating the record-keeping application using a desktop automation tool based on the data to generate the patient record includes storing the patient record in a database.

12. A system for automatically generating a patient record, via a user-oriented record-keeping application, based on vocalized expression, comprising:
a processor; and
computer-readable memory coupled to the processor and storing processor-executable instructions that, when executed, configure the processor to:
generate a first transcript using a first transcription model based on an audio signal indicative of the vocalized expression,
generate second transcript using a second transcription model based on the audio signal, independently of the first transcription model,
determine a third transcript based on the first and second transcripts by using a large language model,
use the third transcript and a machine learning model to determine data suitable for generating the patient record, and
operate the record-keeping application using a desktop automation tool based on the data to generate the patient record.

13. The system of claim 12, wherein to determine the third transcript based on the first and second transcripts by using a large language model includes to determine the third transcript based on the first and second transcripts by using a plurality of independent large language models.

14. The system of claim 13, wherein to determine the third transcript based on the first and second transcripts by using a plurality of independent large language models includes to
generate a plurality of candidate transcripts using the plurality of independent large language models, each of the plurality of candidate transcripts being uniquely associated with a corresponding one of the plurality of independent large language models; and
generate the third transcript based on the plurality of candidate transcripts.

15. The system of claim 14, wherein to generate the third transcript based on the plurality of candidate transcripts includes to
receive a user input responsive to the plurality of candidate transcripts, and
modify the plurality of candidate transcripts based on the user input to generate the third transcript.

16. The system of claim 15, wherein to generate the third transcript based on the plurality of candidate transcripts includes to use a supervised learning model to process the plurality of candidate transcripts to generate the third transcript, and the processor-executable instructions, when executed, further configure the processor to:
update the supervised learning model based on the user input.

17. The system of claim 14, wherein to generate the third transcript based on the plurality of candidate transcripts includes to use a supervised learning model to process the plurality of candidate transcripts to generate the third transcript.

18. A non-transitory computer-readable medium having stored thereon machine interpretable instructions which, when executed by a processor, cause the processor to perform a computer-implemented method of automatically generating a patient record, via a user-oriented record-keeping application, based on vocalized expression, the computer-implemented method comprising:
receiving an audio signal indicative of the vocalized expression;
generating a first transcript using a first transcription model based on the audio signal;
generating a second transcript using a second transcription model based on the audio signal, independently of the first transcription model;
determining a third transcript based on the first and second transcripts by using a large language model;
using the third transcript and a machine learning model to determine data suitable for generating the patient record; and
operating the record-keeping application using a desktop automation tool based on the data to generate the patient record.

19. The non-transitory computer-readable medium of claim 18, wherein the large language model is a first large language model, and the machine learning model is a second large language model independent of the first large language model.

20. The non-transitory computer-readable medium of claim 18, wherein
generating the first transcript using the first transcription model based on the audio signal includes
generating a first candidate transcript using the first transcription model based on the audio signal,
receiving a first user input responsive to the first candidate transcript, and
modifying the first candidate transcript based on the first user input to generate the first transcript; and
generating the second transcript using the second transcription model based on the audio signal, independently of the first transcription model, includes
generating a second candidate transcript using the second transcription model based on the audio signal,
receiving a second user input responsive to the second candidate transcript, and
modifying the second candidate transcript based on the second user input to generate the second transcript.
